(19) United States
(12) Patent Application Publication
(10) Pub. No.: US 2025/0308638 A1
(43) Pub. Date: Oct. 2, 2025

(54) ONTOLOGY PROPAGATION

(71) Applicant: CANON MEDICAL SYSTEMS CORPORATION, Otawara-shi (JP)

(72) Inventors: Russell HUNG, Edinburgh (GB); Simon FISHER, Edinburgh (GB)

(73) Assignee: CANON MEDICAL SYSTEMS CORPORATION, Otawara-shi (JP)

(21) Appl. No.: 18/617,713

(22) Filed: Mar. 27, 2024

(51) Int. Cl. G16B 50/10; G16B 20/00

(52) U.S. Cl. CPC G16B 50/10; G16B 20/00

(57) ABSTRACT

A medical information processing apparatus comprising processing circuitry configured to receive omics data comprising a plurality of biomolecules and a plurality of associated measured values; receive a first plurality of associations mapping a respective biomolecule to another respective biomolecule; receive ontology data based on the omics data, the ontology data comprising a plurality of ontology terms associated with at least one other ontology term and/or at least one other biomolecule; and assign a value to each of the plurality ontology terms based on the omics data, the ontology data, and the associations between them. A value can be assigned to each of the ontology terms based on a propagation algorithm.

### FIELD

The present invention relates to the characterisation of omics data with ontology annotations, in particular by using algorithms.

### BACKGROUND

The Gene Ontology (GO) is an on-going effort to assign biological roles to genes based on the functions of the gene products (i.e. proteins and functional RNAs) by gathering knowledge from research, laboratory studies and databases. GO terms are frequently used to functionally annotate genes and proteins. The GO describes knowledge in the biological domain with respect to three categories: molecular function, cellular component and biological process. The ontological terms are loosely hierarchical, with child terms being more specialised than parent terms. A term can have more than one parent term.

Transcriptomic approaches which involve the computational analysis of gene expression data are regularly used in biomedical research to understand the role that certain genes may play in certain biological states. For example, transcriptomics analyses may be conducted to understand the relationships between genes and disease classifications, tissue classifications, drug responses, or other phenotypes.

Machine Learning methods are now a routine part of a bioinformatics workflow and are typically used to learn the associations between genes and biological effects. Machine learning methods use algorithms to provide an output, such as a classification, based on input data. When applied transcriptomics, it is common to use gene expression values as feature inputs. A challenge with this approach is that transcriptomics datasets are high dimensional, capturing the expression values for thousands of genes. This can make it difficult to identify the genes that may be associated with a particular biological effect. Common approaches to overcome this include the use of machine learning models such as autoencoders to produce a lower dimensional embedding of the input data, or the use of graph convolution techniques to leverage the information in the scientific literature relating to known interactions among genes so that only known interactions are included in a model.

However, these techniques generally do not integrate knowledge relating to the biological annotations of genes that have been generated by a large body of research. A further limitation with these modelling approaches is that multiple genes often work together to produce a biological effects. The interactions between these genes sometimes are not learned by simple models. A further challenge with black box machine learning models is that even when models perform well, it is not apparent why some genes have a greater association with a biological effect than other genes. There is often a disconnect between the importance of some features (genes) in a certain task and a consideration of their functional role within the associated biological system (explain-ability). Essentially, it is difficult to explain why some genes in isolation are predictive of a given task.

Label propagation algorithms are semi-supervised machine learning algorithms that assign labels to un-labelled data observations in order to classify all of the data observations within a dataset. Other techniques such as message passing (belief propagation) in graph convolutional networks also fall into this category. Recent work has expanded on this principle to enable missing features in a dataset to be filled based on data observations with known features. For example, Rossi et al. “On the unreasonable effectiveness of feature propagation in learning on graphs with missing node features.” Learning on Graphs Conference. PMLR, 2022, describes a technique they term feature propagation to assign features to nodes in a graph by diffusing the known features in the graph.

### FIGURES

FIG. 1 is a schematic diagram of an apparatus according to an embodiment;

FIG. 2 is a schematic diagram of omics data comprising gene expression values for three subjects;

FIG. 3 is a schematic diagram of gene ontological terms structured as a tree;

FIG. 4 is a flow chart illustrating an overview of a method in accordance with an embodiment;

FIG. 5 is a flow chart illustrating a method in accordance with an embodiment; and

FIG. 6A is a schematic diagram of omics data and FIG. 6B is a concatenated graph based on the omics data;

FIG. 7A is a schematic diagram of omics data and FIGS. 7B-7D show a concatenated graph based on the omics data at the beginning, mid-point and end of the graph propagation method respectively.

### DESCRIPTION

Certain embodiments provide a medical information processing apparatus comprising a processing circuitry configured to: receive omics data comprising a plurality of biomolecules and a plurality of measured values, wherein each of the plurality of measured values are associated with a respective biomolecule of the plurality of biomolecules; receive a first plurality of associations based on the omics data, each of the first plurality of associations mapping a respective biomolecule of the plurality of biomolecules to another respective biomolecule of the plurality of biomolecules; receive ontology data based on the omics data, the ontology data comprising a plurality of ontology terms and a second plurality of associations, each of the second plurality of associations mapping a respective ontology term of the plurality of ontology terms to either a respective biomolecule of the plurality of biomolecules or another respective ontology term of the plurality of ontology terms; and assign a value to each of the plurality ontology terms based on the omics data, the ontology data, and the plurality of biomolecule associations.

Certain embodiments provide a medical information processing method comprising: receiving omics data comprising a plurality of biomolecules and a plurality of measured values, wherein each of the plurality of measured values are associated with a respective biomolecule of the plurality of biomolecules; receiving ontology data based on the omics data, the ontology data comprising a plurality of ontology terms and a plurality of associations, each of the plurality of associations mapping each of the plurality of ontology terms to a respective biomolecule of the plurality of biomolecules or a respective ontology term of the plurality of ontology terms; and assigning a value to each of the plurality ontology terms based on the plurality of associations, the plurality of measured values, and a propagation algorithm.

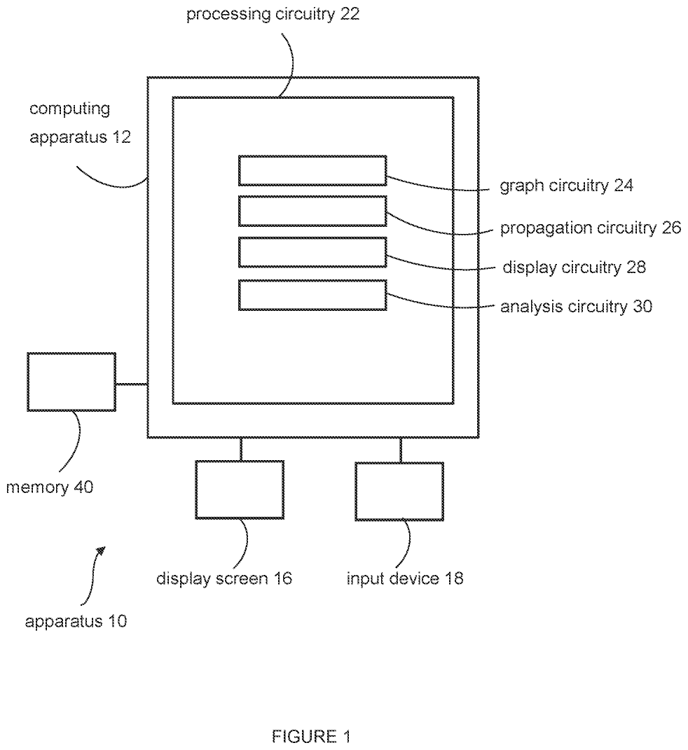

An apparatus 10 according to an embodiment is illustrated schematically in FIG. 1. The apparatus 10 may also be referred to as a medical information processing apparatus. The apparatus 10 is configured to process omics data and ontology data. The apparatus 10 is further configured to display an image based on the omics data and the ontology data.

In other embodiments, the apparatus 10 may be configured to process any appropriate data, which may comprise non-omics data, such as any unordered data. For instance, in some embodiments, the apparatus 10 may be configured to process any data comprising a plurality of variables and a plurality of values, wherein each of the plurality of values is associated with a respective variable of the plurality of variables.

The apparatus 10 comprises a computing apparatus 12, which in this case is a personal computer (PC) or workstation. The computing apparatus 12 is connected to a display screen 16 or other display device, and an input device or devices 18, such as a computer keyboard and mouse. The computing apparatus 12 receives data from memory 40, which may also be referred to as a data store or storage. In alternative embodiments, computing apparatus 12 receives data from one or more further data stores (not shown) instead of or in addition to memory 40. For example, the computing apparatus 12 may receive data from one or more remote data stores (not shown), which may comprise cloud-based storage.

The memory 40 stores omics data 100 which quantifies the amounts of certain biomolecules for one or more subjects. The memory 40 further stores ontology data 90 comprising ontological terms for characterising biomolecules. In other embodiments, the ontology data may be stored in another suitable memory, for example in another apparatus or in a cloud-based memory.

Computing apparatus 12 comprises a processing apparatus 22 for processing data. The processing apparatus 22 comprises a central processing unit (CPU) and Graphical Processing Unit (GPU). The processing apparatus 22 provides a processing resource for automatically or semi-automatically processing omics data sets 100 and an ontology database 110.

The processing apparatus 22 includes a graph circuitry 24 configured to process omics data 100 and an ontology database 110 to produce a graph 140 connecting the omics data to ontology terms from the ontology database, a propagation circuitry 26 configured to propagate values from the omics data to the ontology terms based on the graph 140, a display circuitry 28 configured to display the graph 140 and the values propagated to the ontology terms, and an analysis circuitry 30 to perform downstream analysis on the ontology terms and associated values.

In the present embodiment, the circuitries 24, 26, 28 and 30 are each implemented in the CPU and/or GPU by means of a computer program having computer-readable instructions that are executable to perform the method of the embodiment. In other embodiments, the circuitries may be implemented as one or more ASICs (application specific integrated circuits) or FPGAs (field programmable gate arrays).

The computing apparatus 12 also includes a hard drive and other components of a PC including RAM, ROM, a data bus, an operating system including various device drivers, and hardware devices including a graphics card. Such components are not shown in FIG. 1 for clarity.

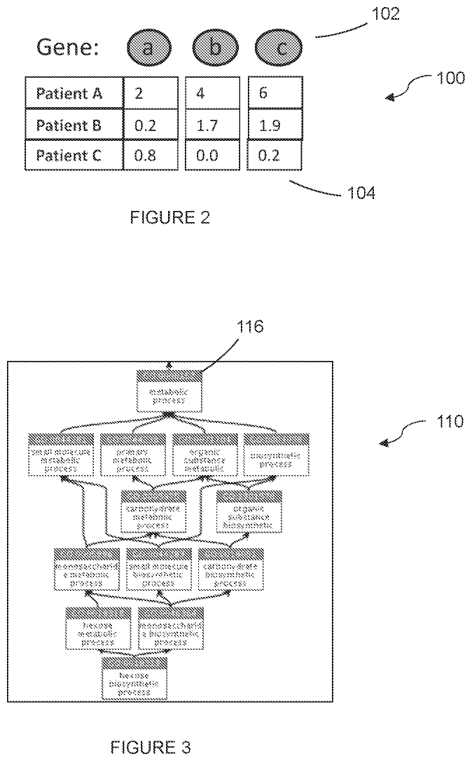

The processing apparatus 22 of FIG. 1 is configured to process omics data 100 illustrated in FIG. 2 and an ontology database 110, part of which is illustrated in FIG. 3. With reference to FIG. 2 the omics data 100 will now be described in further detail. FIG. 2 shows omics data 100 comprising a plurality of biomolecules 102 and a plurality of measured values 104. Each of the plurality of measured values 104 is associated with a respective biomolecule of the plurality of biomolecules 102. The omics data 100 relates to one or more subjects. In the present embodiment, the omics data 100 relates to a plurality of subjects such that each of the plurality of measured values 104 is associated with a respective subject and a respective biomolecule 102. The omics data 100 may be stored in a matrix with the rows of the matrix corresponding to biomolecules 102 and the columns corresponding to subjects, or vice versa. Each of the cells in the matrix are populated by a corresponding value of the plurality of measured values 104.

The omics data 100 may be transcriptomics data, wherein the plurality of biomolecules 102 are genes, and the plurality of measured values 104 are gene expression values. The transcriptomics data may be obtained based on RNA sequencing techniques performed on bulk samples, such as microarray, RT-qPCR and RNA-Seq, or may be obtained based on single cell analysis techniques such as single cell RNA-Seq. In other embodiments, the omics data 100 may be any other suitable type of data such as proteomics data, where the plurality of biomolecules 102 are proteins and the plurality of measured values 104 are protein abundance levels. The proteomics data may be obtained based on techniques such as mass spectrometry. The omics data 100 may be experimental data as part of clinical study or it may be obtained from a bioinformatics database 90 such as the Gene Expression Omnibus.

FIG. 3 shows part of an ontology database 110 for characterizing omics data 100. The ontology database 110 may be structured as a tree and comprises a plurality of ontology terms 116. The ontology database 110 may be obtained from a bioinformatics database 90 such as the Gene Ontology (GO). The GO characterizes genes by mapping the genes (or gene products, e.g. protein or RNA) to ontology terms relating to one of three domains: molecular function, cellular component or biological process. The GO maps a plurality of ontology terms 116 to respective genes using a GO annotation if there is basis for this association in the scientific literature. The ontology terms 116 are organized as nodes in a tree structure, wherein each node has zero or more child nodes. A root node corresponds to the most general level of an ontology term 116, with each descending level of child nodes corresponding to a more specific ontology term 116. All nodes have one or more parents, except for the root node which has no parent. There are three root nodes, each corresponding to one of molecular function, cellular component or biological process. For example, as shown in FIG. 3, the ontology term 116 corresponding to hexose biosynthetic process is the most specific term, and the ontology term 116 corresponding metabolic process is the most general term. The GO employs a transitivity principle, such that annotation of a gene by one ontology term 116 implies annotation to all parents of the ontology term 116. In alternative embodiments, the ontology data may be obtained from any other bioinformatics database 90 that provides ontological annotations for genes or gene products.

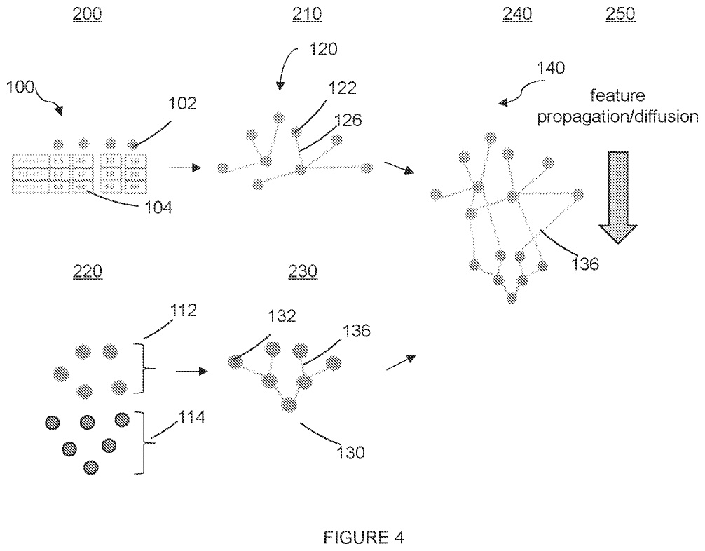

Turning to FIG. 4, an overview of a method to characterize the omics data 100 performed by the processing apparatus 22 will now be described.

At stage 200, the processing apparatus 22 receives omics data 100 for one or more subjects. The omics data 100 comprises a plurality of biomolecules 102 and a plurality of measured values 104, each of the plurality of values associated with a respective biomolecule 102 of the plurality of biomolecules 102.

At stage 210, the processing apparatus 22 constructs a biomolecule graph 120 based on the omics data 100. Each of the first plurality of nodes 122 of the biomolecule graph 120 correspond to a respective biomolecule of the plurality of biomolecules 102. The nodes 122 are connected by edges 126 based on known or predicted interactions between respective pairs of biomolecules of the plurality of biomolecules 102 that are obtained from bioinformatics database 90. The processing apparatus 22 assigns values to the first plurality of nodes 122 based on the corresponding plurality of measured values 104.

At stage 220, the processing apparatus 22 accesses an ontology database 110 and determines a plurality of child ontological terms 112 based on the ontology database 110 and the omics data 100. Based on the child ontological terms 112, the processing apparatus 22 then mines the ontology database 110 to determine a plurality of parent ontological terms 114. The combined set of plurality of child ontology terms 112 and the plurality of parent ontology terms 114 will be referred to as a plurality of ontology terms 116.

At stage 230, the processing apparatus 22 constructs an ontology graph 130 based on the plurality of ontology terms 116. The ontology graph 130 comprises a second plurality of nodes 132, each of the second plurality of nodes 132 corresponding to a respective ontology term 116 of the plurality of ontology terms 116. The second plurality of nodes 132 are connected by edges 136 based on ontological associations between the corresponding ontology terms 116.

At stage 240, the processing apparatus 22 combines the biomolecule graph 120 with the ontology graph 130 to form combined graph 140 by connecting the first plurality of nodes 122 with the second plurality of nodes 132 based on ontological associations between the corresponding child ontology terms 112 and biomolecules 102.

At stage 250, the processing apparatus 22 assigns an initial value of zero to each of the second plurality of nodes 132. The processing apparatus 22 then performs a feature propagation or diffusion algorithm on the combined graph 140 to propagate values from the first plurality of nodes 122 to the second plurality of nodes 132. Once the propagation algorithm has finished running, the final values 142 assigned to each of the second plurality of nodes 132 are output alongside the corresponding ontology terms 116.

The resulting ontology terms 116 and associated values can be used in downstream analysis tasks, such as a machine learning tasks. Since the ontology terms 116 are unambiguously associated with a specific biological function, process or component, they enable the omics dataset to be readily interpreted from a functional perspective. The ontology terms 116 and associated values provide a functional representation of the omics data 100.

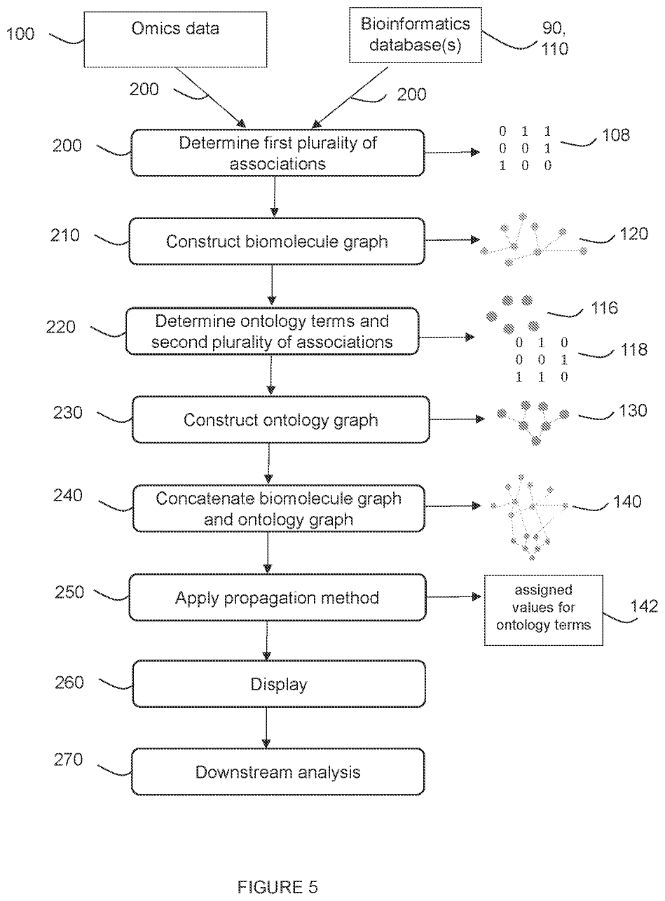

Turning to FIG. 5, the method to characterize the omics data 100 will now be described in further detail.

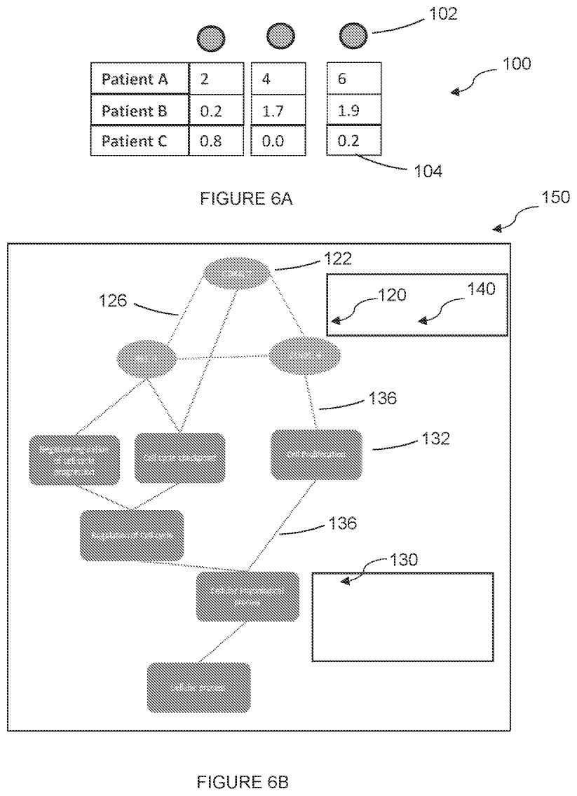

At stage 200, the graph circuitry 24 receives omics data 100 for one or more subjects from memory 40 or from any suitable data store. The omics data 100 comprises a plurality of biomolecules 102 and a plurality of measured values 104, each of the plurality of measured values 104 associated with a respective biomolecule of the plurality of biomolecules 102. In the present embodiment, the omics data 100 is transcriptomics data, wherein the plurality of biomolecules 102 are genes, and the plurality of measured values 104 are gene expression values. FIG. 6A shows omics data 100 for a plurality of patients A, B and C.

The graph circuitry 24 also accesses one or more bioinformatics databases 90 which are stored on the memory 40 or any suitable data store. The graph circuitry 24 determines a first plurality of associations 108 based on the omics data 100 and the one or more bioinformatics databases 90. Each of the first plurality of associations 108 map one of the plurality of biomolecules 102 to another of the plurality of biomolecules 102 based on a known or predicted associations between those biomolecules or their products as described in the one or more bioinformatics databases 90. For example, if the omics data 100 is transcriptomics data comprising and the first plurality of biomolecules 102 are genes, the first plurality of associations 108 may be based on the interactions between the protein products of those genes

The one or more bioinformatics databases 90 may be a database that stores knowledge using a network, or knowledge graph, such as the STRING (Search Tool for the Retrieval of Interacting Genes/Proteins) or KEGG (Kyoto Encyclopedia of Genes and Genomes) database. In such a database, biomolecules are represented by nodes which are connected to each other by edges if the scientific literature indicates that have or are predicted to have an interaction. An interaction may be a physical interaction or an indirect (functional) association. Edges may be weighted based on the strength of that interaction or based on the confidence in how likely that interaction is thought to be true.

The first plurality of associations 108 may be stored as an unweighted adjacency matrix, wherein the both the rows and columns of the adjacency matrix correspond to the plurality of biomolecules 102, and each of the elements in the matrix are equal to one when there is an association between the corresponding biomolecules 102, and equal to zero when there is no association between the corresponding biomolecules 102. If the adjacency matrix is based on a database such as STRING that defines weighted edges between genes, then a threshold may be chosen such that the first plurality of associations 108 only registers an association between a pair of biomolecules if the corresponding proteins in the STRING database have an connecting edge with a weight greater than or equal to the threshold.

In addition to the above, or alternatively, if the omics data 100 comprises data relating to a plurality of subjects, the first plurality of associations 108 may be based on a correlation in the measured values associated with each of the plurality of biomolecules 102.

At stage 210, the graph circuitry 24 constructs a biomolecule graph 120 based on the plurality of biomolecules 102 and the first plurality of associations 108. The graph 120 comprises a first plurality of nodes 122, each of the first plurality of nodes 122 corresponding to a respective biomolecule of the plurality of biomolecules 102. Respective pairs of the first plurality of nodes 122 are connected by an edge 126 if the first plurality of associations 108 indicates an association between the respective pair of corresponding biomolecules 102.

The graph circuitry 24 assigns values to the first plurality of nodes 112 based on the corresponding plurality of measured values 104. If the omics data 100 relates to a plurality of subjects, a vector of corresponding measured values 104 may be assigned to each of the first plurality of nodes 112. Alternatively, a separate biomolecule graph 120 may be constructed for each of the subjects.

At stage 220, the graph circuitry 24 accesses the ontology database 110 from memory 40 or from any suitable data store and determines a plurality of child ontology terms 112 and a second plurality of associations 118 based on the omics data 100 and the ontology database 110. Each of the plurality of child ontological terms 112 correspond to the most specific term (i.e. the lowest level term) that can be associated with a respective biomolecule of the plurality of biomolecules 102. Each of the second plurality of associations 118 map a respective ontology term 112 to a respective biomolecule 102. The second plurality of associations 118 may be stored as an adjacency matrix, with both the rows and columns each corresponding to the plurality of biomolecules 102 and the plurality of child ontology terms 112, and with an entry in each of the cells equal to one if there is an association, and zero if there is no association. Since many genes/proteins may relate to the same biological process or component, there may be more than one biomolecule 102 associated with a respective child ontology term 112. Furthermore, since a respective gene may have more than one function, there may be more than one child ontology term 112 associated with a respective biomolecule 102. In some embodiments, for example, where the ontology database 110 is the GO ontology, each of the child ontological terms 112 corresponds to only one domain of the GO ontology. In other embodiments, the ontology database 110 may have a different structure.

The graph circuitry 24 then mines the ontology database 110 to determine a plurality of parent ontology terms 114 based on the plurality of child ontology terms 112. Each of the plurality of parent ontology terms 114 is a parent of one or more child ontology terms 112 and/or one or more parent ontology terms 114. The second plurality of associations 118 is extended to define the associations between respective pairs of parent ontology terms 114 and respective pairs of parent 114 and child ontology terms 112.

If the second plurality of associations 118 is stored as an adjacency matrix, then the rows and the columns are each expanded to define the parent ontology terms 114. The cells corresponding to respective pairs of ontology terms 112, 114 are then assigned the value one if there is an association, and zero if there is not. The total combined set of child ontology terms 112 and parent ontology terms 114 will be referred to as ontology terms 116. If the ontology database is the GO, the second plurality of associations 118 define a mapping that preserves the structure of the GO.

At stage 230, the graph circuitry 24 constructs an ontology graph 130 based on the plurality of ontology terms 116 and the second plurality of associations 118. The ontology graph 130 comprises a second plurality of nodes 132 that are connected by edges 136 is correspondence with the second plurality of association 118.

At stage 240, the graph circuitry 24 combines the biomolecule graph 120 with the ontology graph 130 produce a combined graph 140. The graph circuitry 24 connects the first plurality of nodes 122 with the second plurality of nodes 132 with edges 136 based on the mappings between the plurality of biomolecules 102 and the child ontology terms 112 defined in the second plurality of associations 118. The connectivity of the combined graph 140 may be stored as an adjacency matrix which is a combination of the respective adjacency matrices for the first plurality of associations 108 and the second plurality of associations 118.

If the omics data 100 relates to a plurality of subjects and a single biomolecule graph 120 is used to hold the information for the plurality of subjects, such that each of the first plurality of nodes 122 is assigned a vector of values, the graph circuitry 24 constructs one combined graph 140. Alternatively, if the omics data 100 relates to a plurality of subjects and a plurality of biomolecule graphs 120 are constructed, each of the biomolecule graphs 120 corresponding to a respective a subject, the graph circuitry 24 constructs a plurality of combined graphs 140, each of the plurality of combined graphs 120 corresponding to a respective a subject.

At the end of stage 240, the graph circuitry 24 passes the combined graph 140 to the propagation circuitry 26.

FIG. 6B illustrates an example of a combined graph 140 which is output from stage 240. The combined graph 140 is based on the omics data 100 illustrated in FIG. 6A, which relates to a plurality of subjects, patients A, B and C. The combined graph 140 comprises biomolecule graph 120 and ontology graph 130. Each of the first plurality of nodes 122 of the biomolecule graph 120 correspond to the genes CDK4, RB1 and CCND2 respectively. The values assigned to each of the nodes 122 correspond to the plurality of measured values 104 associated with patient A. It can be seen from the structure of the ontology graph 130 the nodes 132 corresponding to “negative regulation of cell cycle progression”, “cell cycle checkpoint”, and “cell proliferation” are the child ontology terms 112, and the remaining nodes 132 correspond to the parent ontology terms 114.

At stage 250, the propagation circuitry 26 receives the combined graph 140 and assigns a value of zero to each of the second plurality of nodes 132. The propagation circuitry 26 then applies a propagation algorithm to propagate values from the first plurality of nodes 122 to the second plurality of nodes 132 based on the structure of the combined graph 140. The algorithm repeats a number of iterations until the algorithm converges, i.e. the values assigned to each respective node of the second plurality of nodes 132 converge to a respective limit.

The propagation algorithm may be a feature propagation algorithm which reconstructs the missing features (i.e. the values for each of the second plurality of nodes 132) by propagating the known features of the combined graph (i.e. the values assigned to the first plurality of nodes 122). The algorithm by Rossi et al referred to previously herein is one example which is suitable for undirected graphs such as combined graph 140. The algorithm operates in the following way: for each iteration of the algorithm, the values assigned to each of the second plurality of nodes 132 are updated based on a matrix multiplication between a matrix based upon the adjacency matrix representing the combined graph 140 and a vector representing the values assigned to the first plurality of nodes 122 and second plurality of nodes 132. At the end of each iteration, the values assigned to the first plurality of nodes 122 are reset to their initial values. Therefore, only the values assigned to the second plurality of nodes 132 change at the end of each iteration until the algorithm reaches convergence. Alternatively, any other suitable feature propagation algorithm may be applied.

The feature propagation algorithm may be a label propagation algorithm. A label propagation algorithm is one of the semi-supervised machine learning algorithms that assigns labels to un-labelled data observations in order to partition classification of data observations within dataset.

Alternatively, the propagation algorithm may be a belief propagation, or message passing algorithm that treats the combined graph 140 as a graph convolution network A belief propagation algorithm is a message-passing algorithm used predominantly in probabilistic graphical models. It is employed to compute the marginal distribution of each hidden node in the graph, given some evidence. The marginal distribution can be determined based on a plurality of transition probabilities. Each of the plurality of transition probabilities define associations between a respective biomolecule and ontology term. A transition probability effectively corresponds to a level of confidence that an ontology term is associated with a given biomolecule. In one embodiment, a belief propagation algorithm such as a sum-product algorithm is used, wherein transition probabilities are summed and multiplied as they pass through the combined graph 140 to compute a marginal distributions at each of the second plurality of nodes 132. In embodiments where the combined graph 140 is a tree, convergent solutions can be derived using sum-product algorithm.

At the end of stage 250, the propagation circuitry 26 outputs the combined graph 140 with the final values assigned to each of the second plurality of nodes 132. The propagation circuitry also outputs the ontology terms 116 and the corresponding assigned values 142.

If the omics data 100 relates to a plurality of subjects, the propagation circuitry 26 outputs a set of ontology terms 116 assigned values 142 for each of the subjects. If there is a respective combined graph 140 for each subject, the propagation circuitry 26 performs the algorithm on each separate combined graph 140 and outputs all of the combined graphs 140. If there is a single combined graph 140 representing all subjects, the propagation circuitry 26 may perform the propagation algorithm on the single combined graph 140. The feature propagation algorithm described herein is suitable for this purpose since it is applicable to graphs with vector-valued features.

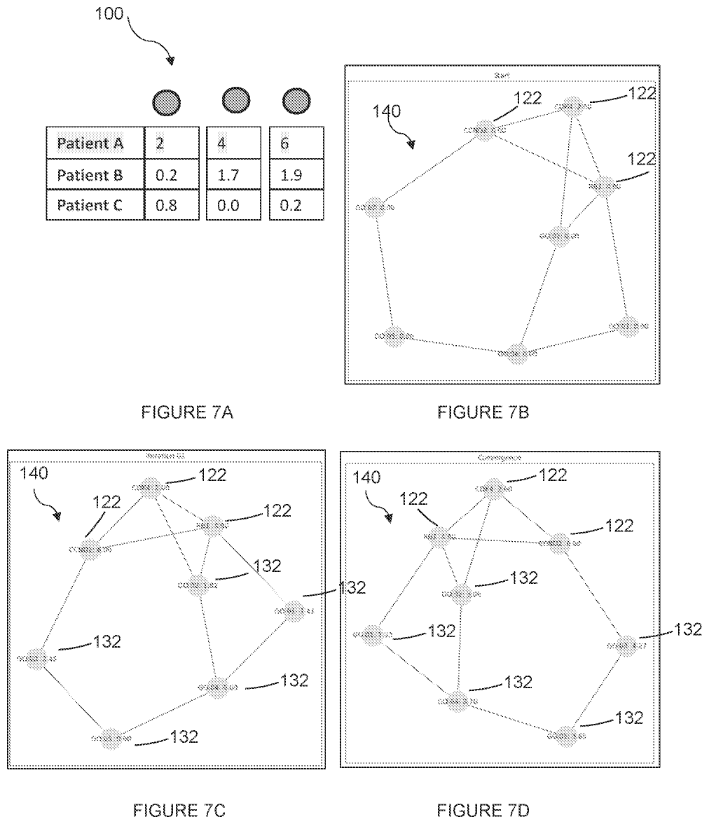

Turning to FIG. 7, this figure illustrates a combined graph 140 based on the omics data 100 of FIG. 7A at the start (FIG. 7B), mid-point (FIG. 7C) and end (FIG. 7D) of stage 250 respectively. FIG. 7B shows each of the first plurality of nodes 122 are assigned values in accordance with the omics data 100 for patient A and each of the second plurality of nodes 132 assigned with the value zero. FIG. 7C shows the combined graph 140 after one iteration of the algorithm. FIG. 7D shows the combined graph 140 after the values propagated to each of the second plurality of nodes 132 have converged.

In the present embodiment, the combined graph 140 is structured as a tree. There are therefore no loops in the tree, which allows the propagation algorithm to reach convergence. In other embodiments, the combined graph 140 may have a different structure which comprises loops. In these embodiments, the propagation algorithm will not completely converge. The algorithm will instead be run until the change in each of the values assigned to each of the second plurality of nodes is below a threshold value.

In the present embodiment, values are assigned to the first plurality of nodes 122 at stage 210, however, in other embodiments, values may be assigned to the first plurality of nodes at the beginning of stage 250.

With reference to FIG. 5, at stage 260, the display circuitry 28 renders an image 150 based on the combined graph 140 and the ontology terms 116 and the corresponding assigned values 142. An example image 150 of the combined graph 140 is illustrated in FIG. 6B. The image 150 may be part of a patient-clinical decision support system to allow a clinician to inspect a dataset relating to a patient. The image 150 may further the assigned values 142 and corresponding ontology terms 116. A ranking of the ontology terms 116 may be displayed based on their corresponding assigned values 142. If the omics data 100 corresponds to more than one subject, a panel may be shown which allows a user to switch between respective images 150 for each of the subjects.

With reference to FIG. 5, at stage 270, the analysis circuitry 30 receives the ontology terms 116 and the assigned values 142 and performs downstream analysis based on the ontology terms 116 and the corresponding assigned values 142.

The downstream analysis may comprise one or more machine learning tasks that are be routinely applied to omics data. For example, the machine learning task may be directed to disease/tissue classification, outcome/phenotype predictions, and predictions relating to drug response or risk of cancer recurrence based on using omics data 100 as an input. The machine learning task may be performed using algorithms such as logistic regression, support vector machine, random forest, a fully connected neural network or any other machine learning algorithm suitable for a classification or prediction task. However, rather than using the raw omics data 100 as an input, the ontology terms 116 and associated values can be used instead. Advantageously, the plurality ontology terms 116 and assigned values 142 integrate the information provided in the omics data 100 but have a significantly reduced dimensionality in comparison. This means that a machine learning algorithm will be able to process input data based on the plurality ontology terms 116 with reduced computational cost.

A subset of the plurality of ontology terms 116 may be selected as an input to a machine learning task based on the hierarchical position of the respective ontology terms 116 and/or the value assigned to the respective ontology terms 116. The hierarchical positioning of the ontology terms 116 provides a way for feature selection to be performed in a straightforward manner.

Downstream analysis may also be performed by relating the plurality of ontology terms 116, or the subset of the plurality of ontology terms 116, with a particular disease state or biological phenotype that corresponds to the omics data.

Advantageously, the use of ontology terms 116 and associated assigned values 142 in place of the raw omics data 100 in a downstream task allows for the link between the data relating to a subject and a classification based on that data to be more easily explained and interpreted with respect to biology.

Features of FIG. 4 and features of FIG. 5 may be combined in any suitable combination. In some embodiments, one or more of the stages described above may be omitted, or multiple stages may be combined.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. A medical information processing apparatus, comprising:
processing circuitry configured to:
receive omics data comprising a plurality of biomolecules and a plurality of measured values, wherein each of the plurality of measured values are associated with a respective biomolecule of the plurality of biomolecules;
receive a first plurality of associations based on the omics data, each of the first plurality of associations mapping a respective biomolecule of the plurality of biomolecules to another respective biomolecule of the plurality of biomolecules;
receive ontology data based on the omics data, the ontology data comprising a plurality of ontology terms and a second plurality of associations, each of the second plurality of associations mapping a respective ontology term of the plurality of ontology terms to either a respective biomolecule of the plurality of biomolecules or another respective ontology term of the plurality of ontology terms; and
assign a value to each of the plurality ontology terms based on the omics data, the ontology data, the first plurality of associations and second plurality of associations.

2. The medical information processing apparatus of claim 1, wherein the value is assigned based on a propagation algorithm.

3. The medical information processing apparatus of claim 2, wherein the propagation algorithm is based on one of feature propagation or belief propagation.

4. The medical information processing apparatus of claim 2,
wherein the value assigned to each of the plurality of ontology terms is based on a graph comprising a first plurality of nodes and a second plurality of nodes, each of the first plurality of nodes corresponding to a respective biomolecule of the plurality of biomolecules and each of the second plurality of nodes corresponding to a respective ontology term of the plurality of ontology terms;
wherein the first plurality of nodes are connected based on the first plurality of associations;
wherein the second plurality of nodes are connected based on the second plurality of associations;
wherein the first plurality of nodes and the second plurality of nodes are connected based on the second plurality of associations;
wherein a value is assigned to each of the first plurality of nodes based on the plurality of measured values; and
wherein the value assigned to each of the plurality of ontology terms corresponds to a value propagated to the corresponding node of the second plurality of nodes based on a propagation algorithm performed on the graph.

5. The medical information processing apparatus of claim 4, wherein values of zero are initially assigned to each of the second plurality of nodes.

6. The medical information processing apparatus of claim 4, wherein the propagation algorithm is a belief propagation algorithm, and wherein a value of propagated to each of the second plurality of nodes based upon the computation of a marginal distribution at each of the respective second plurality of nodes, wherein the marginal distribution is determined based upon transition probabilities between a respective node of the first plurality of nodes and a respective node of the second plurality of nodes.

7. The medical information processing apparatus of claim 1, wherein the processing circuitry is further configured to:
perform downstream analysis based on the plurality ontology terms and the values assigned to each of the plurality of ontology terms.

8. The medical information processing apparatus of claim 7, wherein the downstream analysis is a machine learning or modelling method.

9. The medical information processing apparatus of claim 7, wherein a subset of the plurality of ontology terms are selected for downstream analysis.

10. The medical information processing apparatus of claim 8, wherein the subset is based on the values assigned to each of the plurality of ontology terms and/or a hierarchy level of each of the plurality of ontology terms.

11. The medical information processing apparatus of claim 9, wherein the omics data is associated with a biological phenotype, and
wherein the medical information processing apparatus is further configured to:
associate the plurality of ontology terms or the subset of the plurality of ontology terms with the biological phenotype.

12. The medical information processing apparatus of claim 1, wherein the ontology data is structured as a tree.

13. The medical information processing apparatus of claim 1, wherein the first plurality of associations and the ontology data are determined based on information from one or more biological databases.

14. The medical information processing apparatus of claim 1, wherein the propagation algorithm is run in an unsupervised manner.

15. The medical information processing apparatus of claim 1, wherein the propagation algorithm is run until convergence.

16. The medical information processing apparatus of claim 1, wherein the omics data is transcriptomics data or proteomics data and the first plurality of associations are protein-protein interactions.

17. The medical information processing apparatus of claim 16, wherein the transcriptomics data is derived from bulk RNA sequencing or single cell analysis.

18. The medical information processing apparatus of claim 1, wherein the ontology terms are gene ontology terms and the second plurality of associations are based on gene annotations.

19. The medical information processing apparatus of claim 1, wherein the processing circuitry is further configured to:
display the plurality of ontology terms and the values assigned to each of the plurality of ontology terms.

20. The medical information processing apparatus of claim 1, wherein the omics data corresponds to a subject, and where the processing circuitry is further configured to:
diagnose the subject based on the plurality of ontology terms and the values assigned to each of the plurality of ontology terms.

21. A medical information processing method comprising:
receiving omics data comprising a plurality of biomolecules and a plurality of measured values, wherein each of the plurality of measured values are associated with a respective biomolecule of the plurality of biomolecules;
receiving ontology data based on the omics data, the ontology data comprising a plurality of ontology terms and a plurality of associations, each of the plurality of associations mapping each of the plurality of ontology terms to a respective biomolecule of the plurality of biomolecules or a respective ontology term of the plurality of ontology terms; and
assigning a value to each of the plurality ontology terms based on the plurality of associations, the plurality of measured values, and a propagation algorithm.
